(19) United States
(12) Patent Application Publication
(10) Pub. No.: US 2006/0099284 A1
(43) Pub. Date: May 11, 2006

(54) Composition containing an extract of fructus psoraleae for inhibiting anxiety and depression, improving memory and treating dementia

(76) Inventor: Sung-Jin Kim, Seoul (KR)

Correspondence Address: STETINA BRUNDA GARRED & BRUCKER, 75 ENTERPRISE, SUITE 250, ALISO VIEJO, CA, 92656, US

(21) Appl. No.: 11/271,317

(22) Filed: Nov. 10, 2005

(30) Foreign Application Priority Data
Nov. 10, 2004 (KR) 10-2004-0091252

(51) Int. Cl. A61K 36/23

(52) U.S. Cl. 424/777

(57) ABSTRACT

Disclosed is a composition comprising of an extract of Fructus Psoraleae for inhibiting anxiety and depression and improving memory. The composition can be used in medicaments and health care foods for preventing and treating anxiety and depression of modern people suffering from brain impairment caused by environmental factors, such as various stresses, drinking, smoking and so on, improving memory, and preventing and treating dementia.

### CROSS-REFERENCE TO RELATED APPLICATIONS

This application claims benefit under 35 U.S.C. § 119 from Korean Patent Application No. 10-2004-0091252, filed on Nov. 10, 2004, the entire content of which is incorporated herein by reference.

### STATEMENT RE: FEDERALLY SPONSORED RESEARCH/DEVELOPMENT

Not Applicable

### BACKGROUND

1. Field of the Invention

The present invention relates to a composition containing an extract of fructus psoraleae for inhibiting anxiety and depression, improving memory and treating dementia. More specifically, the present invention relates to a pharmaceutical composition and health care foods having the effects of inhibiting anxiety and depression, improving memory, and treating dementia using an extract of fructus psoraleae.

2. Description of the Related Art

It is known that anxiety, depression and memory disorders are closely related with each other. A body of evidence suggests that as a result of memory failure, depression or anxiety may be exhibited (Schmand, B., Jonker, C., Geerlings, M. I., Lindeboom, J., Subjective memory complaints in the elderly, depressive symptoms and future dementia. Br. J. Psychiatry 171, 373-376, 1997; Harwood, D. G., Barker, W. W., Ownby, R. L., Duara, R., Relationship of behavioral and psychological symptoms to cognitive impairment and functional status in Alzheimer's disease; Int. J. Geriatr. Psychiatry 15, 393-400, 2000; Clarnette, R. M., Almeida, O. P., Forstl, H., Paton, A., Martins, R. N., Clinical characteristics of individuals with subjective memory loss in Western Australia: results from a cross-sectional study; Int. J. Geriatr. Psychiatry 16, 168-174, 2001).

In addition, it is reported that anxiety and depression are attributed to the reaction to a start of memory decline rather than a cause of memory decline (Devanand, D. P., Sano, M., Tang, M. X. et al., Depressed mood and the incidence of Alzheimer's disease: what is consensual”? What is controversial? What is practical?; J. Clin. Psychiatry 59, 6-18, 1996; Jorm, A. F., Christensen, H., Korten, A. E., Hendersen, A. S., Jacomb, P. A., Mackinnon, A., Do cognitive complaints either predict future cognitive decline or reflect past cognitive decline? A longitudinal study of an early community sample; Psychol. Med. 27, 91-98, 1997). The interaction of memory disorders and anxiety is known to be bi-directional because anxiety brings about memory loss (Derouesne, C., Lacomblez, L., Thibault, S., LePoncin, M., Memory complaints in young and elderly subjects; Int. J. Geriatr. Psychiatry 14, 291-301, 1999) and memory loss also causes anxiety (Schneider, L. S., Overview of generalized anxiety disorder in the elderly; J. Clin. Psychiatry 57, 34-45, 1996). Accordingly, the importance of this bi-directional effect is that anxiety and memory loss are closely related (Sinoff, G., Werner, P., Anxiety disorder and accompanying subjective memory loss in the elderly as a predictor of future cognitive decline; Int. J. Geriatr. Pychiatry 18, 951-959, 2003). Up to now, benzodiazepine-based drugs have been used as an anti-anxiety agent, but have had side effects, such as anterograde amnesia, movement disorders, mental dysfunction, and confusion. Acetylcholinesterase inhibitors are now used as an antidementia agent, but they are not effective, e.g., tacrine, widely used, showed noticeable side effects such as abdominal cramps, anorexia, nausea, vomiting, diarrhea or the like in one-third of the patients that took it. Other acetylcholinesterase inhibitors, including donepezil, rivastigmine, galantamine or the like, also have a limited usage as a result of side effects such as nausea, vomiting, diarrhea, insomnia, etc. (Harman J. G., Limbird, L. E. Goodman & Gilman's The Pharmacological Basis of Therapeutics, 10th edition, McGraw Hill, 2001). Accordingly, there is a need for a substance capable of curing anxiety and memory disorders that is efficacious and exhibits less side effects than those drugs currently used.

Fructus Psoraleae, also known as Psoraleae Semen, is an herb of dry and ripe fruits from Psoralea Corylifolia L. or Cullen corylifolia (L.) Medicus and has several common names, such as psoraleae, bu gu zhi, scurry pea and Babchi seeds. It contains corylifolin, bavachin, corylifolinin, isobavachalone, bavachromene, and neobavachalcone in flavonoids, psoralen, isopsoralen, psoralidin, isopsoralidin, angelicin, and corylidin in coumarins, and bakuchiol in monoterpines. Fructus psoraleae has various pharmacological actions as follows: it increases calcification of bones useful in treating fractures, osteomalacia, osteoporosis, and so on; in addition, it has various actions on a cardiovascular system to relax the coronary artery, increase cardiac output, and increase myocardial contractility; and, it is known to have a contraceptive effect, an anti-cancer effect, and a hemostatic effect (Jung, B. S., Sin, M. G., Dohaehyangyakdaesajeon, Younglimsa, p 795-796, 1998; Traditional Oriental Medicine Database (TradMed) Natural Products Research Institute (NPRI) of Seoul National University, 1999; Zhu, Y. P. Chinese Materia Medica. Chemistry, Pharmacology and Applications, Harwood Academic Publishers, 1998).

However, an extract of Fructus Psoraleae has not been reported to inhibit anxiety and depression, improve memory or treat dementia.

As a result of studying substances capable of inhibiting anxiety and depression of people suffering from brain impairment caused by environmental factors, such as various stresses, drinking, smoking and so on; improving memory; and treating dementia, we have found that an extract of fructus psoraleae has excellent effects of inhibiting anxiety and depression, improving memory, and treating dementia.

### BRIEF SUMMARY

An object of the present invention is to provide pharmaceutical compositions and health care foods containing an extract of fructus psoraleae having the effects of inhibiting anxiety and depression, improving memory, and treating dementia.

Another object of the present invention is to effectively prevent and treat interactive anxiety, depression, and memory disorders using an extract of fructus psoraleae.

### BRIEF DESCRIPTION OF THE DRAWINGS

The above aspects and features of the present invention will be more apparent by describing certain embodiments of the present invention with reference to the accompanying drawings, in which:

FIG. 1 indicates the anti-anxiety effect of an extract (fraction T) of Fructus Psoraleae (in a staircase test). The value is an average±standard deviation (n=5) and significance to a control group is *: P<0.05.

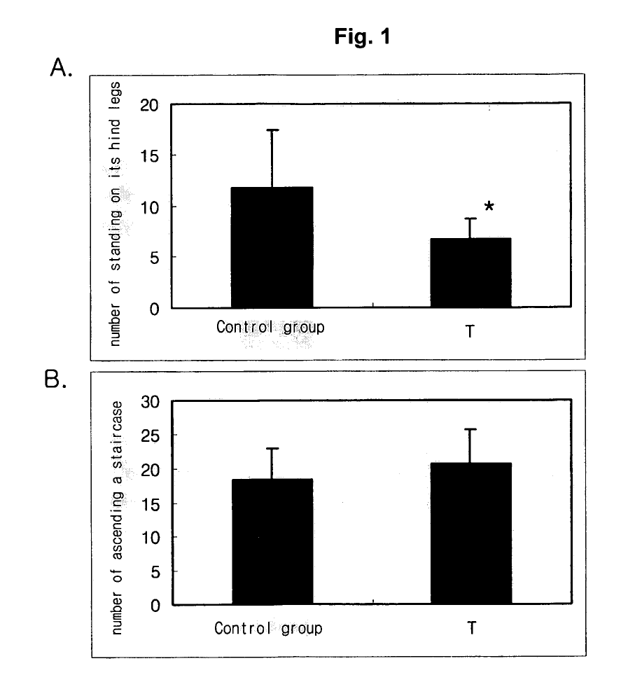

FIG. 2 indicates the anti-anxiety effect of extracts (fractions T, C, CM, M) of Fructus Psoraleae (in an elevated plus maze test). The value is an average±standard deviation (n=5) and significance to a control group is *: P<0.05.

FIG. 3 indicates the anti-anxiety effect of extracts (fractions T, C, CM, M) of Fructus Psoraleae (in an elevated plus maze test). The value is an average±standard deviation (n=5) and significance to a control group is *: P<0.05.

FIG. 4 indicates the memory improvement effect of extracts (fractions T, C, CM, M) of Fructus Psoraleae (in a passive avoidance test). The value is an average±standard deviation (n=5) and significance to a control group is *: P<0.05.

FIG. 5 indicates the dementia treatment effect of extracts (fractions T, C, CM, M) of Fructus Psoraleae (in a passive avoidance test). The value is an average±standard deviation (n=5) and significance to a control group is *: P<0.05.

### DETAILED DESCRIPTION

In order to accomplish the above objects according to an aspect of the present invention, there is provided a composition containing Fructus Psoraleae for improving memory and inhibiting anxiety and depression.

The composition of the present invention for improving memory and inhibiting anxiety and depression contains 0.5-50% by weight of an extract of Fructus Psoraleae.

The extract of Fructus Psoraleae of the present invention may be prepared by a process as follows:

First step: Fructus Psoraleae is extracted with organic solvent, such as C1-4 lower alcohols like methanol, ethanol, etc.; acetone; chloroform; methylene chloride; ether, ethylacetate; and so on, preferably methanol or a mixture of methanol and water ranging from 1:0.2 to 1:1.5, at a temperature of 5° C. to 80° C., preferably 30° C. to 55° C., for a reaction time of 15 minutes to 48 hours, preferably 30 minutes to 12 hours to obtain a lower alcohol soluble fraction containing a large amount of terpenoids and phenolic materials.

Second step: the above lower alcohol soluble fraction is dissolved in a mixture of lower alcohol and water, adjusted to pH 2-4 with acid, and extracted with an equal amount of chloroform to obtain a chloroform soluble fraction.

Third step: a chloroform insoluble fraction is adjusted to pH 9-12 with ammonium hydroxide, extracted with an equal amount of a mixture of chloroform:methanol, and fractionated to obtain a chloroform-methanol soluble fraction, wherein a mixing ratio of chloroform:methanol is preferably in a range of 1:0.1 to 1:1. While a chloroform-methanol soluble fraction contains most alkaloids upon extracting the chloroform insoluble fraction with a mixture of chloroform:methanol, a methanol soluble fraction of a chloroform-methanol insoluble fraction includes quarternary alkaloids and N-oxides.

Fourth step: the chloroform-methanol insoluble fraction is additionally extracted with methanol and fractionated to obtain a methanol soluble fraction.

The present invention provides a composition for improving memory and inhibiting anxiety and depression, which contains a lower alcohol soluble fraction, a chloroform soluble fraction, a chloroform-methanol soluble fraction, and a methanol soluble fraction from the foregoing steps.

Furthermore, the extract of fructus psoraleae of the present invention can be additionally fractionated by a general. fractionation (Harborne J. B., Phytochemical methods: A guide to modern techniques of plant analysis; 3rd Ed. pp 6-7, 1998).

The composition containing the extract of Fructus Psoraleae of the present, invention may further include suitable carriers, excipients, and diluents according to standard methods known within the art.

Carriers, excipients, and diluents that can be included in the composition containing the extract of Fructus Psoraleae of the present invention are lactose, dextrose, sucrose, sorbitol, mannitol, xylitol, erythritol, maltitol, starch, acacia gum, alginate, gelatin, calcium, phosphates, calcium silicates, cellulose, methyl cellulose, microcrystalline cellulose, polyvinyl pyrrolidone, water, methylhydroxybenzoates, propylhydroxybenzoates, talc, magnesium stearate and mineral oils.

The composition containing the extract of Fructus Psoraleae of the present invention can be formulated in the forms of oral formulations such as powder, tablet, capsule, suspension, emulsion, syrup, aerosol; external applications; suppositories; and sterile injections according to methods known within the art.

The dose of an extract of fructus psoraleae can vary with the patient's age, sex, and weight, and may be administered in a dose of 0.1 mg to 500 mg per kilogram of body weight once or several times a day. A dosage of an extract or fractions of Fructus Psoraleae can be increased and decreased depending on administration routes, disease severity, sex, weight, age and so on. Accordingly, the above dose is not intended to limit the scope of the invention in any way.

The composition containing the extract of Fructus Psoraleae of the present invention can be widely used in medicaments, foods, beverages, etc. for improving memory and inhibiting anxiety in the above formulation. Examples of items capable of containing an extract of Fructus Psoraleae are various foods, beverages, gums, teas, vitamin complex supplements, health care foods, etc.

The extract of Fructus Psoraleae of the present invention itself has little toxicity or side effects, thereby it is a medicament capable of being safely taken for a long period of time for the purpose of disease prevention.

The extract of Fructus Psoraleae of the present invention can be added to foods or beverages for the purpose of improving memory and inhibiting anxiety and depression. The extract of Fructus Psoraleae can generally be added to the health food composition of the invention in an amount of 0.1 to 15 percent by weight, preferably 1 to 10 percent by weight, and may be added in a ratio of 1-30 g, preferably 3-10 g relative to a health beverage composition of 100 mL.

The health beverage composition puts no special limitation on liquid ingredients except that it contains the extract of Fructus Psoraleae as an essential ingredient in the indicated ratio, and can comprise various flavoring agents, natural carbohydrates, etc. as an additional ingredient like ordinary beverages. Examples of the above-mentioned natural carbohydrates are ordinary saccharides, such as monosaccharides, for example glucose, fructose, etc; disaccharides, for example maltose, sucrose, etc; and polysaccharides, for example dextrin, cyclodextrin, etc.; and sugar alcohols such as xylitol, sorbitol, erythritol, etc. Other than the above-mentioned flavoring agents, natural flavoring agents (thaumatin, stevia extracts (for example, revaudioside A, glycyrrhizin, etc.)) and synthetic flavoring agents (saccharins, aspartame, etc) can be advantageously used. Said natural carbohydrates are used in an amount of about 1-20 g, preferably about 5-12 g based on the composition of 100 mL of the invention.

In addition, the composition of the invention can include various nutrients, vitamins, minerals (electrolytes), synthetic and natural flavors, colorants and enhancers (cheese, chocolate, etc.), pectic acid and its salts, alginic acid and its salts, organic acids, protective colloidal thickeners, pH modifiers, stabilizers, antiseptics, glycerin, alcohols, and carbonating agents used in carbonated beverages. Moreover, the composition of the present invention can contain pulp for preparing natural fruit juices, fruit beverages and vegetable beverages. These ingredients may be used individually or in combination. The ratio of these additives is not important, but is generally selected in a range of 0 to about 20 parts by weight per 100 parts by weight of the composition of the present invention.

The following examples illustrate the invention in more detail, but are not to be construed as limiting the present invention.

Example 1: Preparation of an Extract of Fructus Psoraleae

Fructus psoraleae (250 g) was cut into fine pieces and extracted three times with 70% methanol (750 mL) using a reflux condenser. The extract was filtered, concentrated under reduced pressure using a rotary evaporator (EYELA N-N Series), and lyophilized to obtain 23.35 g of a crude extract in methanol (fraction T).

In order to fractionate the lyophilized methanol-extract with other organic solvent; the extract (8 g) was dissolved in a mixture of methanol:water (4:1), adjusted to pH 3 with 2M sulfuric acid, and continuously extracted three times with an equal amount of chloroform. It was concentrated under reduced pressure and lyophilized to obtain 1.891 g of a chloroform soluble fraction (fraction C). A water layer was adjusted to pH 10 with ammonium hydroxide and twice extracted with an equal amount of chloroform:methanol (3:1). A soluble fraction in a mixture of chloroform:methanol (3:1) was concentrated under reduced pressure and lyophilized to obtain 0.174 g of a chloroform-methanol soluble fraction (fraction CM). A remaining water layer was extracted with an equal amount of methanol three times, concentrated under reduced pressure, and lyophilized to obtain 6.16 g of a methanol soluble fraction (fraction M). These fractions were used as a sample in the following activity tests.

Experimental Example 1: Anti-anxiety Test (Staircase Test)

1) Method

An anti-anxiety test was carried out as described by Simiand et al. (Simiand, J., Keane, P. E., Morre, M., The staircase test in mice: A simple and efficient procedure for primary screening of anxiolytic agents; Psychopharmacolgy 84, 48-53, 1984). A mouse was put on the bottom of a staircase box (10 cm×45 cm×25 cm) with its tail toward the staircase. And then, the number of steps the mouse ascended and the number of times the mouse stood on its hind legs were measured for 3 minutes, wherein one step means that four feet of the mouse were on the staircase. In order to simplify the observation, the number of times descending the staircase was excluded. After the test, the staircase was swiftly cleaned so as not to stimulate the sense of smell of the next mouse. The fraction T of an extract of fructus psoraleae was orally administered in a dose of 10 mg/kg 60 minutes before the test, wherein all tests were performed from 8 a.m. to 5 p.m.

2) Experimental Results

In the case of the mouse administered with fraction T of Fructus Psoraleae, the number of times standing on its hind legs, a measure of anxiety, decreased by 42.86% compared with a control group. But, there was no significant difference between. a fraction T-administered group and a control group in the number of steps ascended, a measure of exploratory activities. These results suggest that fraction T of fructus psoraleae has a remarkable anti-anxiety action.

Experimental Example 2: Anti-anxiety Test (Elevated Plus Maze Test)

1) Method

An elevated plus maze test was carried out one hour after an extract of fructus psoraleae (fraction T, fraction C, fraction CM, or fraction M) was orally administered to a male ICR mouse (20 g) in a dose of 10 mg/kg. The maze, which is 70cm high, has two open arms (50×10 cm) and two closed arms (50×10×40 cm) crossed (Pellow, S., Chopin, P. H., File, S. E., Briley, M., Validation of open:closed entries in an elevated plus maze as a measure of anxiety in the rat; J. Neurosci. Meth. 14, 149-167, 1985). A mouse was put in the middle of the maze and turned toward the closed arms one hour after the fractions (T, C, CM, M) of Fructus Psoraleae were orally administered in a dose of 10 mg/kg. And then, the number and time of entry into the open arms and the closed arms were measured for 5 minutes.

2) Experimental Results

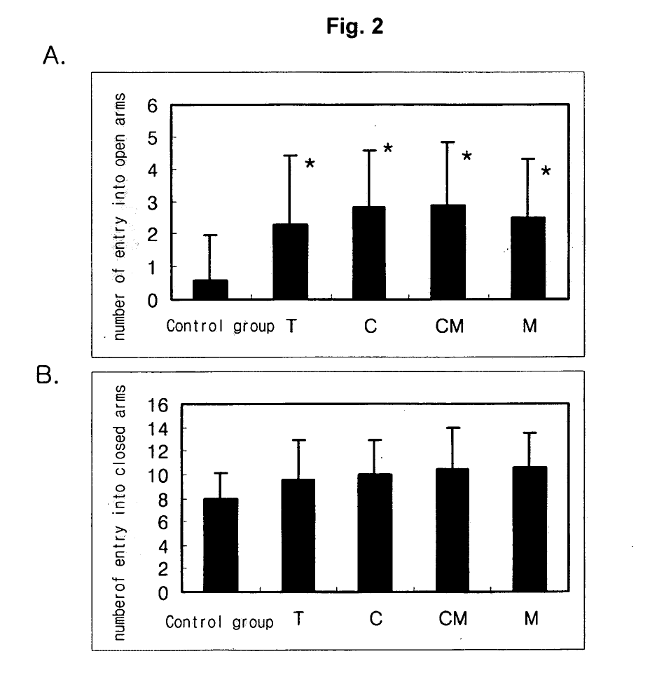

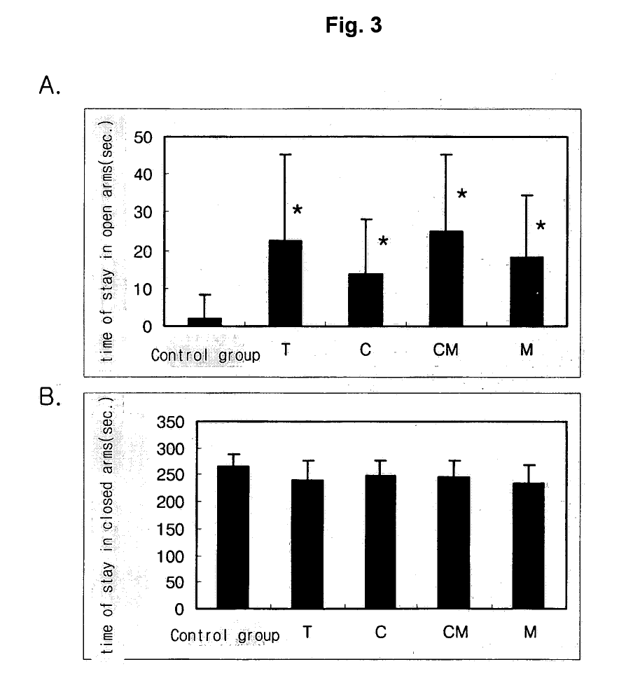

In case of the mouse administered with extracts (T, C, CM, M) of fructus psoraleae, the number of entry into open arms increased by 3.81 times, 4.67 times, 4.76 times, and 4.17 times respectively, as compared with a control group (FIG. 2A). However, there was no significant difference between a fructus psoraleae-administered group and a control group in the number of entry into closed arms (FIG. 2B). In case of the mice administered with extracts (T, C, CM, M) of fructus psoraleae, the time of entry into open arms increased by 10.69 times, 6.50 times, 11.84 times, and 8.62 times respectively, as compared with a control group (FIG. 3A). However, there was no significant difference between a fructus psoraleae-administered group and a control group in the time of entry into closed arms (FIG. 3B). The number of entries into the open arms and the time of stay in the open arms in an elevated plus maze test indicate a lower state of anxiety. The number of entries into the closed arms and the time of stay in the closed arms indicate a measure of exploratory activities. Accordingly, all extracts (T, C, CM, M) of fructus psoraleae have a remarkable anti-anxiety effect.

Experimental Example 3: Passive Avoidance Test: Memory Improvement Test

1) Method

A passive avoidance test was carried out using a Gemini avoidance system (San Diego Instruments, USA) one hour after extracts (fraction T, fraction C, fraction CM, or fraction M) of Fructus Psoraleae were orally administered to a male ICR mouse (20 g) in a dose of 10 mg/kg. The test was modified on the basis of a method by Kumar, et al. and carried out as follows (Kumar, V., Singh, P.N., Muruganandan, A. V., Bhattacharya. Effect of Indian Hypericum perforatum Linn on animal models of cognitive dysfunction; J Ethnopharmacology 72, p 119-128, 2000).

On the first day of a training test, a mouse was put into the bright box to be acclimated for 300 seconds, before moving into a dark box by automatically opening a door. If the mouse moved into the dark box, it was electrically stimulated one second at 0.3 mA. When tested after 24 hours, the mouse was acclimated in the bright box for 300 seconds, prior to moving into the dark box by opening a door. The time to move into the dark box was measured at this time. On the second day, an electrical stimulation was not given to the mouse unless the mouse moved into the dark box for 180 seconds (Mohamed, A. F., Matsumoto, K., Tabata, K., Takayama, H., Kitajima, M., Aimi, N., Watanabe, H., Effects of Uncaria tomentosa total alkaloid and its components on experimental amnesia in mice: Elucidation using the passive avoidance test; J. Pharm. Pharmacol. 52, 1553-1561, 2000).

2) Experimental Results

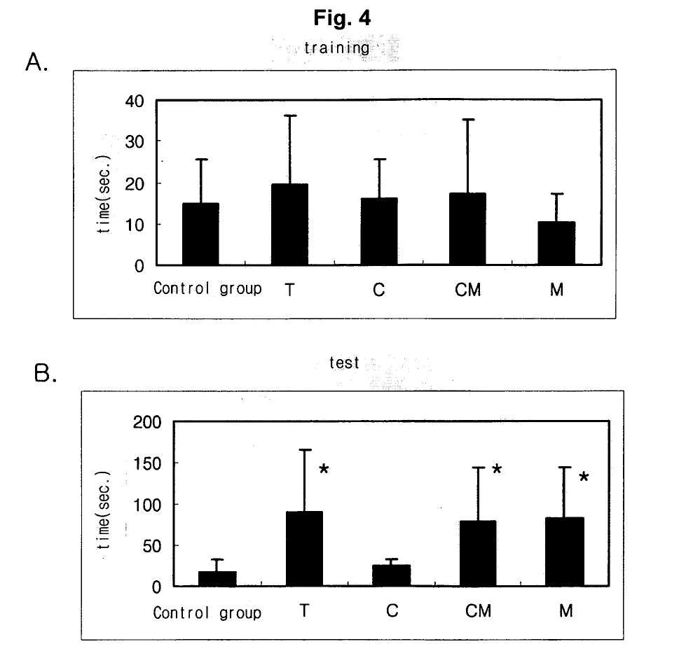

As shown in FIG. 4A, there was no significant difference among experimental groups on the first day of a training test. As shown in FIG. 4B, the mouse administered with fractions T, C, CM, and M of fructus psoraleae showed a remarkable memory improvement effect by 4.93 times, 1.33 times, 4.30 times, and 4.55 times respectively, as compared with the control group.

Experimental Example 4: Passive Avoidance Test: Dementia Treatment Test

1) Method

A passive avoidance test was carried out using a Gemini avoidance system (San Diego Instruments, USA) one hour after extracts (fraction T, fraction C, fraction CM, or fraction M) of Fructus Psoraleae were orally administered to a male ICR mouse (20 g) in a dose of 10 mg/kg. The test was modified on the basis of a method by Kumar, et al. and carried out as follows (Kumar, V., Singh, P. N., Muruganandan, A. V., Bhattacharya, Effect of Indian Hypericum perforatum Linn on animal models of cognitive dysfunction; J Ethnopharmacology 72, p 119-128, 2000).

On the first day of a training test, a mouse was put into the bright box to be acclimated for 300 seconds, prior to moving the mouse into a dark box by automatically opening a door. If the mouse moved into a dark box, it was electrically stimulated one second at 0.3 mA. As soon as a training session was finished, scopolamine was intraperitoneally administered in a dose of 1 mg/kg. At the test after 24 hours, the mouse was acclimated in the bright box for 300 seconds, prior to moving the mouse into the dark box by opening a door. The time to move into the dark box was measured at this time. On the second day, an electrical stimulation was not given to the mouse unless the mouse moved into the dark box for 180 seconds (Mohamed, A. F., Matsumoto, K., Tabata, K., Takayama, H., Kitajima, M., Aimi, N., Watanabe, H., Effects of Uncaria tomentosa total alkaloid and its components on experimental amnesia in mice: Elucidation using the passive avoidance test; J. Pharm. Pharmacol. 52, 1553-1561, 2000).

2) Experimental Results

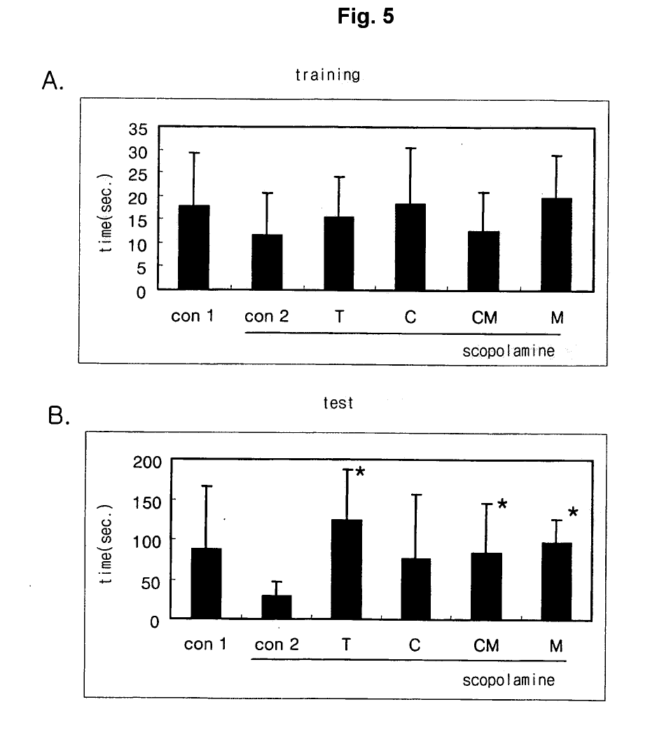

As shown FIG. 5A, there was no significant difference among the experimental groups on the first day of training test. As shown FIG. 5B, the mouse administered with scopolamine to express dementia showed memory failure of 66.6% compared with a control group. However, the mouse administered with fractions T, C, CM, and M of fructus psoraleae showed an excellent memory recovery effect by increasing memory deteriorated by scopolamine by 4.25 times, 2.63 times, 2.90 times, and 3.32 times respectively.

Experimental Example 5: Oral Toxicity Test of Extracts of Fructus Psoraleae

1) Method

30 male ICR mice (20 g) were raised a week in an animal room that had a temperature of 23□, relative humidity of 50%, and illuminance of 150-300 Lux, and then they were assigned to five groups, 5 mice each.

In order to examine the safety of the extracts of Fructus Psoraleae, lethal dose 50% (LD50) was used as an index showing acute toxicity of drugs.

The methanol extract fraction T of Fructus Psoraleae was orally and intraperitoneally administered to each group of 10 mice in a dose of 50 mg/kg, 500 mg/kg, 5,000 mg/kg, 10,000 mg/kg, and 20,000 mg/kg. The change of general symptoms and presence of dead animals were observed for 7 days after administration. The mouse was killed and anatomized to examine internal organs with the naked eye on the seventh day after an administration.

2) Experimental Results

Abnormal findings resulting from the extract fraction T of Fructus Psoraleae were not observed. The lethal dose 50% of an intraperitoneal administration was more than 10 g/kg, and the lethal dose 50% of an oral administration was more than 20 g/kg. There was no specific toxicity on various organs in a biopsy. Accordingly, the extract fraction T of Fructus Psoraleae showed a high level of safety.

Hereinafter, the preparative examples of said pharmaceutical composition will be explained. They are not intended to limit the present invention, but are given only for illustration of the invention.

Preparative Example 1. Tablets

According to the following composition, tablets were prepared by a general tablet preparation.

| | |
|---|---|
| Methanol extract of Fructus Psoraleae | 500.0 mg |
| Lactose | 500.0 mg |
| Talc | 5.0 mg |
| Magnesium stearate | 1.0 mg |

Preparative Example 2. Capsules

According to the following composition, capsules were prepared by the following method.

Extracts of Fructus Psoraleae were screened and mixed with excipients, prior to filling gelatin capsules.

| | |
|---|---|
| Methanol extract of Fructus Psoraleae | 500.0 mg |
| Starch 1500 | 10.0 mg |
| Magnesium stearate BP | 100.0 mg |

Preparative Example 3. Syrup

According to the following composition, syrup was prepared by the following method.

First, white sugar was dissolved in purified water. Thereto paraoxybenzoate, paraoxypropylbenzoate and extracts of Fructus Psoraleae were added and dissolved at 60□, prior to cooling the mixture purified water was added to make 150 mL.

| | | |
|---|---|---|
| Methanol extract of Fructus Psoraleae | 5.0 | g |
| White sugar | 95.1 | g |
| Paraoxybenzoate | 80.0 | mg |
| Paraoxypropylbenzoate | 16.0 | mg |

Preparative Example 4. Solution

The following ingredients were formulated by a general solution preparation, prior to filling a brown bottle purified water was added to make 100 mL.

| | | |
|---|---|---|
| Methanol extract of Fructus Psoraleae | 500.0 | mg |
| Isomerized sugar | 20.0 | g |
| Antioxidant | 5.0 | mg |
| Methyl paraoxybenzoate | 2.0 | mg |

Preparative Example 5. Powder

The following ingredients were mixed by a general powder preparation, put into a bag and sealed to prepare powder.

| | |
|---|---|
| Methanol extract of Fructus Psoraleae | 50.0 mg |
| Lactose | 100.0 mg |
| Talc | 5.0 mg |

Preparative Example 6. Injection

The following ingredients were formulated by a general injection preparation to fill an ample of 2.0 mL, prior to sterilizing the formulation distilled water was added to make 2.0 mL.

| | |
|---|---|
| Methanol extract of Fructus Psoraleae | 50.0 mg |
| Antioxidant | 1.0 mg |
| Tween 80 | 1.0 mg |

In addition, health foods were prepared by the following method:

Brown rice, barley, sweet rice, and Job's tears were gelatinized and dried, prior to roasting the dried cereals were made into a powder with particle size of 60 mesh using a crusher according to a known method. Black soybeans, black sesame, and perilla also were steamed and dried, prior to roasting the dried seeds were made into a powder with particle size of 60 mesh using a crusher according to a known method.

Cereals, seeds, and dry extracts of fructus psoraleae prepared above were blended in the following ratios by weight to make granules:

| | |
|---|---|
| brown rice | 30% |
| Job's tears | 15% |
| barley | 20% |
| perilla | 7% |
| black soybeans | 8% |
| black sesame | 7% |
| dry powder of extracts of Fructus | 3% |
| Psoraleae | |
| Ling chiu mushrooms | 5% |
| rehmanniae radix | 5% |

A composition containing an extract of Fructus Psoraleae has the effects of improving memory and inhibiting anxiety and depression when used in people at risk of brain impairment resulting from various environmental stresses and improving the memory of people with poor memory, including dementia patients.

The foregoing embodiment and advantages are merely exemplary and are not to be construed as limiting the present invention. The present teaching can be readily applied to other types of apparatuses. Also, the description of the embodiments of the present invention is intended to be illustrative, and not to limit the scope of the claims, and many alternatives, modifications, and variations will be apparent to those skilled in the art.

What is claimed is:

1. A pharmaceutical composition comprising an extract of Fructus Psoraleae for inhibiting anxiety and depression, improving memory, and preventing and treating dementia.

2. The composition as claimed in claim 1, comprising an extract of Fructus Psoraleae in an amount of about 0.5 percent by weight to about 50 percent by weight based on the total composition.

3. The composition as claimed in claim 1, wherein the extract of Fructus Psoraleae is obtained by extracting Fructus Psoraleae with an organic solvent selected from a group consisting of C1-4 lower alcohols, acetone, chloroform, methylene chloride, ether, and ethyl acetate.

4. The composition as claimed in claim 3, wherein the extract of Fructus Psoraleae is dissolved in a mixture of methanol and water, adjusted to pH 2 to 4 with acid, further extracted with an equal amount of chloroform, and fractionated to obtain a chloroform soluble fraction.

5. The composition as claimed in claim 3, wherein the extract of Fructus Psoraleae is dissolved in a mixture of methanol and water, adjusted to pH 2 to 4 with acid, and further extracted with an equal amount of chloroform, followed by adjusting a chloroform insoluble fraction to pH 9 to 12 with ammonium hydroxide to be extracted with an equal amount of a chloroform-methanol mixture, and fractionated to obtain a chloroform-methanol soluble fraction.

6. The composition as claimed in claim 3, wherein the extract of Fructus Psoraleae is dissolved in a mixture of methanol and water, adjusted to pH 2 to 4 with acid, and extracted with an equal amount of chloroform, followed by adjusting a chloroform insoluble fraction to pH 9 to 12 with ammonium hydroxide to be extracted with an equal amount of a chloroform-methanol mixture, wherein a chloroform-methanol insoluble fraction is further extracted with methanol and fractionated to obtain a methanol soluble fraction.

7. The composition as claimed in claim 1, comprising a further component selected from the group consisting of carriers, excipients, or diluents.

8. The composition as claimed in claim 1, wherein said composition has a formulation selected from the group consisting of a powder, tablet, capsule, suspension, emulsion, syrup, aerosol, external application, suppository, and sterile injection.

9. A health care food comprising:
a) an extract of Fructus Psoraleae having the effect of inhibiting anxiety and depression, improving memory, and treating dementia; and
b) at least one acceptable food additive.
